(19) United States
(12) Patent Application Publication
(10) Pub. No.: US 2019/0256932 A1
(43) Pub. Date: Aug. 22, 2019

(54) STR BASED RAPID PERSONAL IDENTIFICATION

(71) Applicant: Huseyin ONAY, Izmir (TR)

(72) Inventor: Huseyin ONAY, Izmir (TR)

(21) Appl. No.: 16/345,733

(22) PCT Filed: Oct. 27, 2016
(86) PCT No.: PCT/TR2016/050406

§ 371 Date: Apr. 29, 2019

(51) Int. Cl. C12Q 1/6888; C12Q 1/686; C12Q 1/6806

(52) U.S. Cl. CPC C12Q 1/6888; C12Q 1/686; C12Q 2537/143; C12Q 2600/16; C12Q 2600/156; C12Q 1/6806

(57) ABSTRACT

A genetic identification kit which provides rapid results and includes a small number of short tandem repeat (STR) regions. A genotyping method with the genetic identification kit, including the following steps: forming a panel comprising dyes allowing an examination in a genetic analyzer and marking primers, obtaining a primary mixture by mixing indicators used for reading genetic analysers, obtaining a PCR master mix mixture, mixing an isolated DNA molecule with the primary mixture and the PCR master mix mixture to obtain a DNA mixture, subjecting the DNA mixture to a multiplex polymerase chain reaction (PCR), examining STR regions in the genetic analyzer, and determining a size range of indicators used.

### CROSS REFERENCE TO THE RELATED APPLICATIONS

This application is the national phase entry of International Application No. PCT/TR2016/050406, filed on Oct. 27, 2016, the entire contents of which are incorporated herein by reference.

### TECHNICAL FIELD

The invention is related to a genetic identification kit which provides rapid results and comprises a small number of short tandem repeat (STR) regions.

### BACKGROUND

The human DNA is one of the most stable biological materials known which comprises several personal identification regions. Although 99.9% of our DNA is the same, the 0.1% difference between individuals allows clear differentiation between people. Although these different regions on the DNA, have started to be used in areas that are susceptible to diseases, the efficiency of DNA in identification studies has been realized and it has started to be used in this area.

Following the examination of some special regions in a person's DNA and the mapping out of a genetic profile of a person DNA has been used for many years in both clinic genetics and forensic medicine. A genetic based profiling test can provide an identification test that is nearly 100% precise. One of the most frequently used methods is the certain genotyping of the Short Tandem Repeat (STR) regions on especially DNA. 50% of our genome is formed of repeating sequences. These repeating regions can have any length and can be classified according to the length of the main sequence that repeats. These regions are called satellite DNA's. The STR regions that are also called microsatellite DNA's are short repeat sequences and they comprise repeating sequences between 2-7 nucleotides. Not only can the polymorphic structure of these regions can separate two people from each other, but they can also differentiate the chromosomes received from a mother or a father in a person. There are two other reasons besides this powerful differentiation ability, for STR indicators to be used in DNA based identification. STR can be easily amplified by means of a Polymerase Chain Reaction (PCR) and they are present in all chromosomes including X and Y chromosomes. The genotyping of the STR region, in other words identification following the showing of repeat numbers in that region via molecular genetic methods, is carried out according to the repeat numbers obtained from that region. The copy of a STR region obtained from the mother and the father is called “allele”. If the repeat numbers in the DNA received from the mother and the father is different, the person has an informative indicator, in other words the person is heterozygote. If the repeat number of the mother and father, in other words the alleles are the same, this is not an informative indicator and the person is a homozygote in terms of indication. Generally the genotyping process is carried out using fluorescent indication primers that are not found in a repeat region.

The genetic profiles obtained by genotyping STR regions, have been accepted as the most significant evidence in courts in the past years. The most important reason for this is that, genetic profiling is data belonging to said person, which cannot be mimicked and can be controlled when desired. During the genetic profiling carried out using sixteen STR regions, the probability of two people to have the same profile is 7.52739×10⁻¹⁶ and the cumulative exclusion probability is 99,9994372%. However these tests that have been used comprehensively in the genetic field until today, have not been used in fields where personal identification is crucially important.

STR based identification kits are already being used in hospitals in evaluating the presence of medical situations such as the presence of the maternal cell contamination during prenatal identification cases. During tests carried out using such kits available in the market, 16 STR regions are examined that have been internationally accepted. Moreover such kits are not produced domestically but are imported from abroad.

Although STR based systems are used in personal identification for medical purposes, they are not used in for security purposes. In fields where advanced personal identification is required one of the most important problems if for a person to be correctly identified. The usage of molecular genetics methods occupy an important place for said identification to be carried out correctly.

When the patent examples regarding the prior art is examined, two patent documents can be cited as reference, which are CN104031989A and EP2055787A1. The patent numbered EP2055787A1 belongs to Hennessy and Green, where a kit has been designed according to the invention which allows analysis on isolated DNA molecules using D16S539, D18S51, D19S433, D21S11, D2S1338, D3S1358, D8S1179, FGA, THO1, VWA and D1S1656 indicators and 6-FAM™, VIC®, NED™ ve PET® fluorescent dyes. A kit which comprises CSF1 PO, D3S1358, D5S818, D7S820, D8S1179, D13S317, D16S539, D18S51, D21S11, FGA, THO1, TPDX, VWA, D2S1338, D19S433, Amelogenin, SE33, DYS19, DYS385a/b, DYS389I/II, DYS390, DYS391, DYS392, DYS393, DYS438, DYS439, DYS437, DYS448, DYS456, DYS458, Y GATA C4 (DYS635) and Y GATA H4 indicators comprising isolated fluorescent dyes such as 6FAM, VIC, NED, PET, JOE, 5FAM, TET, ROX, HEX, ve TAMARA using DNA samples which are again isolated is described in the patent document numbered CN104031989A. Both kits allow STR analysis using similar methods. Due to the abundance of indicators in the above mentioned kits and fluorescent dyes that are used, the analysis time takes longer in comparison to our invention.

The present products in the market are analyzed using the Multiplex PCR feature. Due to the abundance of the STR regions examined by these kits, the cost is high and the analysis time is long. As these kits comprise regions besides the STR regions that need to be examined, and as similar samples that are sold in the market are the same with each other, product selectivity is limited. One of the most important problems here is that such genetic tests take a long time to be carried out. Genetic testing takes around 4 hours even with the fastest planning nowadays and this restricts their usage in other sectors.

The invention aims to produce a panel for the identification kit used in STR based research in our country, which shall be specific to the Turkish community, and to reduce the analysis time to 30 minutes. As there is no genetic identification kit which comprises the STR features of the Turkish Community available in the market, this poses an obstacle against research to be carried out regarding this issue. The STR indicators within these kits are for general usage and due to the abundance of the regions to be examined said kits are expensive and take a long time to provide results.

### SUMMARY

The invention is a STR based genetic identification kit, in which STR indicators whose heterogeneity ratios in the Turkish Community are high, are used, which can carry out analysis in a shorter period of time, which comprises a small number of STR regions and which provides rapid results.

A fast and reliable profiling test based on genetics, which is different to all other biometric methods, which can provide nearly 100% identification has been developed according to the invention for fields in which an advanced level of personal identification is required. The invention is a rapid genetic test which enables the DNA based identification of a person and it aims to finalize the genetic test at such a short period of time that has not been achieved anywhere else in the world according to routine usage. Another aim of the invention is to create one of the fastest personal genetic identification algorithm's in the world with the cooperation of university-industry and to be able to use this algorithm in the banking sector. Moreover a prototype product which can be used for cosmic rooms found in the state organizations and institutions, for military applications such as rocket launching units, nuclear plants, and civil applications such as pharmaceutical development units and special units of Telco operators has been developed by means of the invention.

The product that has been developed is a quick result product for medical purposes that can be used in relation to personal identification. It is a product which can particularly make it easier to obtain information regarding the success of a transplant operation such as a marrow transplant operation, before and after said operation. Besides this, our product which allows the determination of cell contamination from the mother to the baby also allows the determination of forensic identification in situations (natural disasters such as earthquakes and floods and terrorist activities), where personal identification is required. As the regions comprised in our product, comprises the regions which enable to characterize the Turkish Community genetically, it allows faster analysis and personal identification in comparison to other products. This allows the analysis to be finalized in a shorter period of time.

### BRIEF DESCRIPTION OF THE DRAWINGS

FIG. 1A shows sizes of D3S1358 (D3), D7S820 (D7) and CSF1PO;

FIG. 1B shows sizes of Amelogenin (AML), D16S539 (D16) and D13S317 (D13);

FIG. 1C shows a size of D18S51 (D18);

FIG. 1D shows a size of FGA indicators; and

FIG. 1E shows a size of GeneScan™ 500 LIZ™ dye Size Standard.

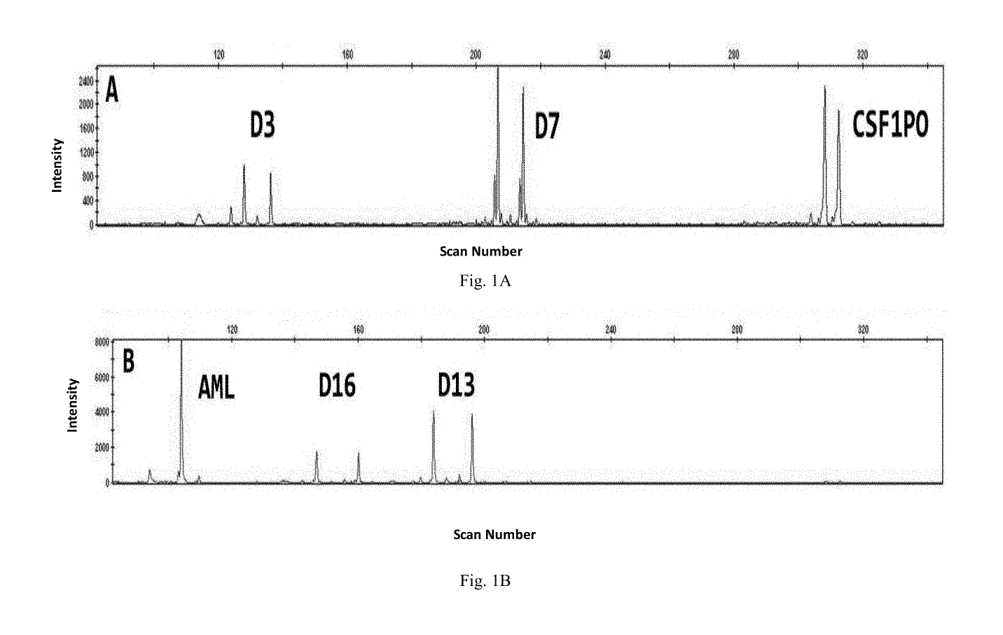

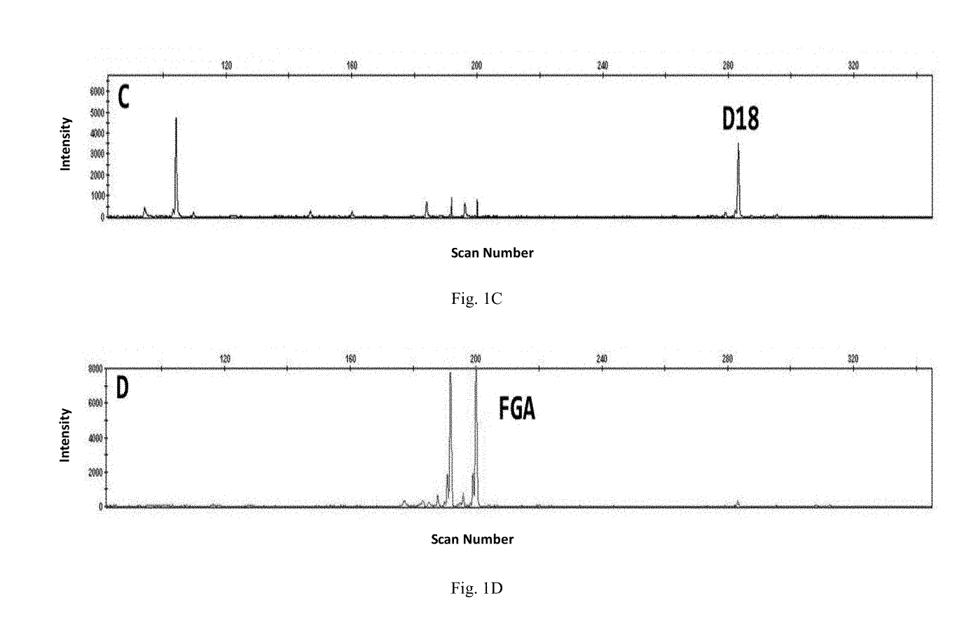

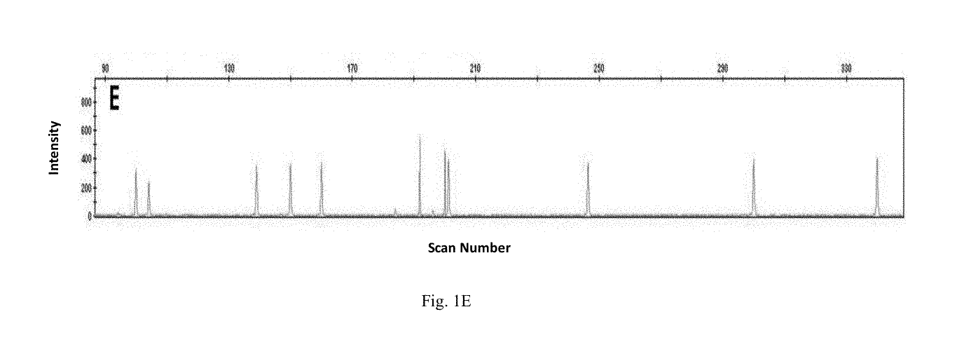

### DETAILED DESCRIPTION OF THE EMBODIMENTS

The invention provides STR analysis using DNA molecules that have been isolated from blood or tissue. The DNA molecule that has been obtained is mixed with primary mixture of the invention and the PCR Master Mix mixture, is first of all subjected to a PCR phase. The PCR process defines the successive processes that are used in order to increase the targeted regions of DNA. The increasing of the desired region of DNA forms the basis of the detection step following said increasing of the desired region. The desired region of DNA can be increased in desired numbers and form by using PCR. The regions of STR indicators desired by the invention can be increased rapidly. A kit which comprises a small number of indicators having high differentiation regarding STR genotyping has been designed. This kit which has at least 75% differentiation ability and provides short PCR products, is important in terms of rapid genotyping. D3S1358, D7S820, CSF1PO, D13S317, D16S539, D18S51, FGA and Amelogenin (sexual indicators) indicators are used for the invention which has been carried out by using 4 fluorescent colours in reading genetic analyzers. A panel is formed comprising 6-FAM, VIC, NED and PET dyes which allow examination in a genetic analyzer and primers are marked. These indicators are mixed in the ratios given in Table 1 and a primary mixture is therefore obtained. A PCR Master Mix Mixture is obtained at ratios mentioned in Table 2. The primary mixture and the PCR master mix mixture of the invention is mixed with isolated DNA molecule and a multiplex PCR process which allows co-amplification with the primers marked on a single DNA molecule is carried out.

TABLE 1
Primary Mixture Ratios

| Indicator | Amount | Indicator | Amount |
|---|---|---|---|
| D3S1358: | 3-6 μl | D16S539: | 3-7 μl |
| D7S820: | 10-13 μl | D18S51: | 1-4 μl |
| CSF1PO: | 4-7 μl | FGA | 8-12 μl |
| D13S317 | 4-7 μl | Amelogenin | 7-10 μl |

TABLE 2
PCR Master Mix Mixture Ratios

| Component | Amount |
|---|---|
| Master Mix | 32-37 μl |
| ddH₂O | 4-7 μl |
| MgCl₂ | 1-4 μl |
| Taq DNA | 0.2-1.2 μl |
| Polymerase | |

The size range of the indicators within the framework of standard protocols using GeneScan™ 500 LIZ™ dye Size Standard in an ABI PRISM® 3130 genetic analyzer in order to examine STR regions following a multiplex PCR process.

What is claimed is:

1. A genetic identification kit, comprising dyes and Short Tandem Repeat (STR) indicators, wherein the genetic identification kit performs an identification by using a DNA molecule having been isolated from a blood or a tissue, and the DNA molecule is Short Tandem Repeat (STR) based.

2. A genotyping method with the genetic identification kit according to claim 1, comprising the following steps:
forming a panel comprising the dyes for examination in a genetic analyzer and marking primers,
obtaining a primary mixture by mixing the STR indicators used for reading the genetic analyzer,
obtaining a PCR master mix mixture,
mixing the DNA molecule with the primary mixture and the PCR master mix mixture to obtain a DNA mixture,
subjecting the DNA mixture to a multiplex polymerase chain reaction (PCR),
examining STR regions in the genetic analyzer, and
determining a size range of the STR indicators used.

3. The genotyping method according to claim 2, wherein, the STR indicators comprise indicators of D3S1358, D7S820, CSF1PO, D13S317, D16S539, D18S51, FGA and a sexual indicator of Amelogenin.

4. The genotyping method according to claim 3, wherein the primary mixture is formed by mixing 3-6 μl of D3S1358, 10-13 μl of D7S820, 4-7 μl of CSF1PO, 4-7 μl of D13S317, 3-7 μl of D16S539, 1-4 μl of D18S51, 8-12 μl of FGA and 7-10 μl of Amelogenin.

5. The genotyping method according to claim 2, wherein, the PCR master mix mixture is obtained by mixing a master mix required for standard PCR, ddH₂O of Nuclease-free/Ultra Pure water, MgCl₂ and Taq DNA Polymerase of a DNA polymerase enzyme.

6. The genotyping method according to claim 2, wherein, a separation power of each of the STR indicators is at least 75%.

7. The genotyping method according to claim 2, wherein, the panel comprising the dyes of 6-FAM, VIC, NED and PET is formed to mark the primers.
